(19) United States
(12) Patent Application Publication
(10) Pub. No.: US 2003/0088229 A1
(43) Pub. Date: May 8, 2003

(54) Absorbent article with bimodal acquisition layer

(76) Inventors: Andrew Baker, Lawrenceville, GA (US); Saundra Naughton, Marietta, GA (US)

Correspondence Address: Patrick A. Doody, Esq., Hunton & Williams, Suite 1200, 1900 K Street, NW, Washington, DC, 20006-1109, US

(21) Appl. No.: 09/985,453

(22) Filed: Nov. 2, 2001

(51) Int. Cl. A61F 13/15; A61F 13/20

(52) U.S. Cl. 604/385.101; 604/378; 604/384

(57) ABSTRACT

A disposable absorbent garment including a topsheet, a backsheet, a storage layer, and an acquisition layer is disclosed. The acquisition layer lies between the topsheet and the storage layer, and includes at least two yarns oriented generally parallel with the longitudinal axis of the article, with an acquisition channel between each adjacent pair of yarns. The acquisition channels provide free volume for temporary fluid storage until the fluids can be absorbed by the storage layer and allow fluids to move to the longitudinal ends of the article. The yarns store fluids until they are absorbed, and wick fluids away from the topsheet to keep the user dry. Each yam may have properties specifically tailored to provide particular benefits depending on each yarn's location in the garment. The absorbent garment using such an acquisition layer is comfortable, highly absorbent, and economical to produce.

### FIELD OF THE INVENTION

The present invention relates generally to absorbent garments. Particularly, it relates to absorbent garments with an acquisition layer or transfer layer disposed between the topsheet and storage layer or absorbent core to assist in distributing incoming fluids.

### BACKGROUND OF THE INVENTION

Traditionally, disposable absorbent articles, such as infant diapers or training pants, adult incontinence products, feminine care products and other such products, are constructed with a moisture-impervious outer backing sheet (or “backsheet”), a moisture-pervious body-contacting inner liner sheet (or “topsheet”), and a moisture-absorbent core (or “absorbent core”) sandwiched between the liner sheet and the backing sheets.

In general, some of the most important performance attributes of an absorbent article are functional capacity, rate of absorption, and core stability in use. Absorption under load, or AUL, of the absorbent core is one measure of functional capacity and the rate at which fluid absorption occurs. The AUL is a function of several variables, including the geometric layout of the absorbent core and the physical and chemical properties of the absorbent core materials.

The absorbent core of the garment typically comprises two main functional layers. The first layer is an acquisition layer, sometimes called a fluid handling layer, transfer layer, or surge layer, and the second layer is called the storage layer. The acquisition layer is typically provided to serve a variety of functions that address common problems with absorbent articles. The absorbent core is usually wrapped in a tissue material that maintains the absorbent core's shape and prevents absorbent material from escaping, but allows essentially uninhibited passage of fluids.

In a typical absorbent article, fluids strike the article in localized regions (insult points), and at relatively high volumes, known as the surge rate, compared with the absorption rate of the article. As the fluid strikes this localized region, the absorbent at that point becomes saturated and unable to absorb the incoming fluid. The acquisition layer must be able to hold all of the unabsorbed excess fluid until it can be absorbed by the storage layer, otherwise the excess fluid will remain in contact with the users skin until it is absorbed. Excess fluid may also leak out of the article before the absorbent core has been able to absorb it. The measure of how well an article can handle incoming fluid is known as the article's surge capacity, which is ideally relatively high. It also is desirable to provide a relatively thin acquisition layer to improve the comfort and fit of the article. Therefore, the use of a thick acquisition layer to absorb the unabsorbed excess fluid would not provide an ideal way of handling the fluid. Instead, the prior art has attempted to provide thin acquisition layers that have high surge capacities, which are obtained by promoting rapid distribution of the fluid away from the insult point and throughout the layer.

Unabsorbed excess fluid tends to redistribute itself radially away from the insult point by natural hydraulic pressure before it is absorbed by the storage layer. Many articles have an elongated shape, so it is desirable to redirect the fluid flow along the length of the article where it may be absorbed by the unwetted portions of the storage layer. It is also desirable to prevent radial flow towards the side edges of the article where fluids may leak out of the article. Ideally, then, the acquisition layer accelerates fluid flow in the longitudinal direction and retards fluid flow in the lateral direction.

The prior art has attempted to create acquisition layers that help redistribute fluids along the length of the article. For example, U.S. Pat. No. 5,669,895, issued to Murakami et al., discloses an absorbent article that uses a “rapid distribution strip” as an acquisition layer. Murakami discloses a grid of rhomboidal compressed regions, in which the rhombuses are elongated in their longitudinal direction. This “screen” of compressed material provides increased longitudinal movement of fluids due to the improved capillary wicking action that occurs in the fibers of the compressed region. In the center of each rhomboid is a longitudinally extending embossed shape that prevents lateral flow of fluid within the layer. See also, PCT. WO 86/01378, issued to Kamstrup-Larsen, et al., which discloses an absorption body having longitudinal channels formed along the length of the article to promote longitudinal fluid flow.

Another problem addressed by the acquisition layer is the problem of “gel blocking.” Many modern absorbent articles use an absorbent core made of super-absorbent polymers (SAP), fluff pulp, or a combination of the two. These cores are desirable because they can typically be made thinner than other cores, which improves comfort and fit of the article. Such super-absorbent materials are generally polymeric gelling materials that are capable of absorbing and retaining, even under moderate pressure, large quantities of liquid, such as urine and body wastes, relative to their weight. When the super-absorbent material in the absorbent core is wetted, it swells and forms a gel.

Gel formation can block liquid transmission into the interior of the absorbent core, however resulting in a phenomenon called “gel blocking.” Gel blocking prevents liquid from rapidly flowing or wicking past the swollen “blocking” particles of super absorbent polymer, causing portions of a partially hydrated core to become inaccessible to multiple doses of liquids, such as urine, water and saline solutions. Further absorption of liquid by the absorbent core must then take place via a diffusion process within the polymer gel. This is typically much slower than the rate at which liquid is applied to the core. Gel blocking often leads to leakage from the absorbent article well before all of the absorbent material in the core is fully saturated. In order to improve the performance of the SAP, the liquid being absorbed in the absorbent structure must be transported to unsaturated super absorbent material. Furthermore, as the super absorbent material absorbs the liquid, it must be allowed to swell into a free volume so as to maintain a capillary structure within the absorbent core to distribute liquid.

In order to overcome problems associated with gel blocking, prior art acquisition layers have attempted to provide rapid distribution of fluid away from the point of initial liquid contact. Distributing large quantities of liquids throughout a large area of the absorbent core increases absorption rates and minimizes gel blocking during absorption of multiple doses of liquid.

Another use for acquisition layers is to prevent rewetting of the topsheet. Rewetting occurs when fluids absorbed by the storage layer return to the topsheet, causing the topsheet to become wet and uncomfortable. A wet topsheet can also lead to rashes and infections. Ideally, the acquisition layer provides a one-way conduit for fluids to be absorbed into the storage layer without being released back out. A properly-working acquisition layer will prevent contact between the topsheet and the storage layer, thus preventing rewetting. For example, U.S. Pat. No. 5,902,757 issued to Stern et al. discloses a multi-layered absorbent core that uses a hydrophobic upper layer and a hydrophilic lower layer. The upper layer captures fluids, which are subsequently wicked into the lower layer to isolate the moisture from the wearer. Rewet may be prevented by providing an acquisition layer that physically separates the topsheet from the storage layer, and that is resilient enough that it will maintain this separation after repeated compressions and deformations of the article.

A problem with conventional acquisition layers is that they are often made from airlaid fluff pulp material or carded thermal bond nonwoven material having a relatively low basis weight. Although a low basis weight generally results in a high fluid volume handling capacity, such materials are costly to ship to manufacturers because they have a high volume. A higher density material would cost less to ship to a manufacturer per unit of production.

Furthermore, conventional acquisition layers are generally homogeneous materials that provide a single mode of fluid acquisition. These materials may be compressed in regions to provide slightly different properties in the compressed regions, and as such produce multiple-modes of fluid acquisition. Although the prior art has disclosed acquisition layers with variations in properties, it is difficult to manipulate a single homogeneous acquisition layer to provide large differences in the properties in different locations in the garment to provide a truly bimodal acquisition layer. Selective manipulation of portions of the acquisition layer also increases the manufacturing cost of the article.

Other, non-homogeneous acquisition layers have been disclosed, but these require additional sheet layers, which are manipulated to form pleats into which bundles of fibrous material are placed to act as longitudinal distribution members. For example, U.S. Statutory Invention Registration No. H1511 discloses an acquisition layer in which bundles of fibers or bundles of fibrous yarns are placed in longitudinally extending pleats of a second topsheet that is attached directly to the storage layer. These articles are relatively expensive to produce because each article requires about twice as much topsheet material as a conventional article, in addition to the cost of the fibrous materials, and require additional manufacturing steps, such as pleating and gluing.

Therefore, it is highly desirable to provide an absorbent article with a relatively thin acquisition layer that provides improved surge capacity and longitudinal redistribution of fluids, thereby providing improved leakage prevention, SAP efficiency and absorption rate. It also is desirable to provide an absorbent article that has improved rewet resistance, and the ability to recover from deformations that occur during use. These requirements are particularly desirable in garments made with thin absorbent cores that are intended to improve comfort, fit, and wearability without sacrificing the liquid absorption characteristics of the absorbent article.

Furthermore, it is desirable to provide an absorbent article having reduced shipping costs and reduced material costs, such that it is relatively inexpensive to manufacture. It also is desirable to provide an absorbent article having an acquisition layer that has widely varying properties in different regions of the garment.

The present invention, as defined by the preferred embodiments, is designed to overcome the foregoing and other deficiencies of prior art absorbent garments while providing a thin absorbent core made from a bimodal acquisition layer and a storage layer.

### SUMMARY OF THE INVENTION

Features of the invention can be achieved by a disposable absorbent article comprising a liquid permeable topsheet operably attached to a liquid impermeable backsheet. A multi-layered absorbent core is disposed between the topsheet and backsheet. The absorbent core has an acquisition layer facing the topsheet, and a storage layer between the acquisition layer and the backsheet. In one exemplary embodiment, the invention is shaped as a feminine care product. In another exemplary embodiment, the invention is shaped as a diaper. In ye t another exemplary embodiment, the invention is shaped as an adult incontinence product.

The acquisition layer is comprised of a plurality of yarns with acquisition channels between the yarns. The yarns and channels are arranged generally parallel to one another and may be oriented along the longitudinal axis of the article. The yarns may be arranged with gaps or openings to permit some lateral flow between the acquisition channels. These openings may be located along the diagonal axes of the acquisition layer. The yarns may also be oriented as a series of concentric rings, which may have gaps to permit the passage of fluid therethrough.

These and other features and advantages of the preferred embodiments will become more readily apparent upon reading the detailed description of the preferred embodiments of this invention in conjunction with the following drawings.

### BRIEF DESCRIPTION OF THE DRAWINGS

FIG. 1 is a planar view of an embodiment of the present invention depicted in the fully-flattened position, with the topsheet partially cut-away to show the interior of the garment;

FIG. 2 is a cross-sectional view of the article of FIG. 1, as viewed across reference line A-A;

FIG. 3 is an embodiment of the present invention with the topsheet removed;

FIG. 4 is another embodiment of the present invention with the topsheet removed;

FIG. 5 is a cross-sectional view of another embodiment of the present invention;

FIG. 6 is a cross-sectional view of yet another embodiment of the present invention; and, FIG. 7 is an isometric view of an embodiment of the present invention in the shape of a diaper, depicted with the seams opened and the garment in the fully-flattened position, with the topsheet partially cut-away to show the interior of the garment.

### DETAILED DESCRIPTION OF THE PREFERRED EMBODIMENTS

As used herein, the terms “absorbent garment,” “garment,” “absorbent article,” and “article” are interchangeable terms that refer to products that absorb and contain exudates. More specifically, these terms refer to products that are placed against or in proximity to the body of the wearer to absorb and contain the various exudates discharged from the body. A non-exhaustive list of examples of absorbent garments includes diapers, diaper covers, disposable diapers, training pants, feminine hygiene products and adult incontinence products. The term “disposable,” in reference to absorbent articles, refers to absorbent garments that are intended to be discarded or partially discarded after a single use (i.e., they are not intended to be laundered or otherwise restored or reused). The term “unitary,” in reference to absorbent articles, refers to an absorbent garment that has essentially a single structure (i.e., it does not require separate manipulative parts such as a cover and an insert).

The absorbent article of the preferred embodiments can be used with all of the foregoing classes of absorbent garments, without limitation, whether disposable, unitary or otherwise. These classifications are used interchangeably throughout the specification, but are not intended to limit the claimed invention. The invention will be understood to encompass, without limitation, all classes and types of absorbent garments, including those described above.

As used herein, the longitudinal axis or longitudinal dimension of the article is the dimension corresponding to the front-to-rear dimension of the user, and the lateral axis or lateral dimension of the article is the dimension corresponding to the side-to-side dimension of the user.

In general terms, the article of the present invention is comprised of a topsheet, a backsheet, and an absorbent core. The topsheet is placed against the user's body, and is generally liquid-pervious to allow fluids to penetrate into the absorbent core, although portions of the topsheet may be liquid-impervious. The backsheet is generally liquid-impervious, to prevent fluid in the absorbent core from being released from the garment, but may be partly or wholly gas-pervious to provide the article with breathability to enhance user comfort.

The absorbent core of the present invention is comprised of at least an acquisition layer and a storage layer whereby the acquisition layer and storage layer maybe comprised of a single layer containing both acquisition and storage components. Preferably, the absorbent core is as thin as possible in order to improve the comfort and appearance of the garment. The importance of thin, comfortable garments is disclosed in U.S. Pat. 5,098,423 to Pieniak et al., which is herein incorporated by reference in its entirety for all purposes and in a manner consistent with the present invention. The acquisition layer is comprised of a plurality of yarns with acquisition channels between the yarns, and the acquisition layer provides improved surge handling capacity, fluid redistribution, and rewet prevention. The storage layer absorbs and holds fluids that enter the article. The storage layer or both the storage layer and the acquisition layer may be enveloped in a liquid-pervious tissue wrap to stabilize the layer or layers.

The acquisition channels in the acquisition layer provide free volumes for accommodating surges of fluid and contribute to transferring the fluids along the longitudinal dimension of the article. The yarns may also retard the lateral flow of fluid to the sides of the absorbent article. The topsheet, yarns, acquisition channels and storage layer may be selected to transfer fluid from the topsheet to the storage layer, while retarding the opposite flow of fluid, and thereby prevent rewet.

The plurality of yarns may have edge yarns near the edges of the absorbent core and center yarns near the center of the absorbent core. The center yarns may have different properties than the edge yarns. the edge yarns may be selected to provide additional wet strength to the absorbent core, may be imbued with skin care products, and may have greater wicking properties than the center yarns. The center yarns may be provided with greater acquisition properties than the edge yarns.

The yarns may be constructed with various diameters, and the diameters of some yarns may be different from the diameters of other yarns. In various embodiments, the yarns may have a diameter of about 1 millimeter to about 6 millimeters, or more preferably a diameter of about 2 millimeters to about 5 millimeters. The yarns may also have a diameter of about 3 millimeters to about 4 millimeters. The diameters of the yarns in the center regions of the absorbent core may be greater than the diameters of the yarns in the edge regions of the absorbent core.

Similarly, the widths of the acquisition channels may be varied, and some acquisition channels may be wider than others. In various embodiments, the acquisition channels may have a width of about 1 millimeter to about 6 millimeters, or more preferably a width of about 2 millimeters to about 5 millimeters. The acquisition channels may also have a width of about 3 millimeters to about 4 millimeters. The widths of the acquisition channels in the center region of the absorbent core may be greater than the widths of those in the edge regions of the absorbent core.

The yarns may, in various embodiments, be made from polyethylene, polyester, polyurethane or polyacrylic synthetics. The yarns may be attached to the storage layer by hot melt adhesive glue along at least a portion of each yarn's length.

The yarns may have one or more plies, which may each comprise any number of filaments. In various embodiments, the yarns may have a yarn denier of about 1000 to about 6000, or more preferably of about 2000 to about 5000, or even more preferably of about 4100 to about 4150. In various embodiments, the filaments making up the yarns may have a filament denier of about 1.00 to about 14.00, or more preferably of about 5.00 to about 10.00, or even more preferably of about 7.00 to about 7.25.

The storage layer may, in one embodiment, be a composite of fibers and super absorbent polymer. The storage layer, or the entire absorbent core, may be encased in an at least partially liquid permeable tissue wrap. In an embodiment in which a tissue wrap encases the storage layer, but not the yarns, the topsheet may be joined to the tissue wrap in one or more of the acquisition channels to create topsheet channels.

In still another embodiment, the topsheet may be joined to the backsheet around the perimeter of the article to hold the absorbent core in place without it being joined to either the topsheet or the backsheet.

In one preferred embodiment, the present invention is in the form of a feminine care product. In another preferred embodiment, the present invention is in the form of a diaper. In yet another preferred embodiment, the present invention is in the form of an adult incontinence product.

These features, and other features, functions and uses of the present invention, are described in greater detail herein. For clarity, features of the present invention that are depicted in more than one Figure are shown with the same reference number in all Figures.

For simplicity, the preferred embodiments of the invention are generally described in terms of a disposable feminine hygiene article 10, such as shown in FIG. 1. It should be understood, however, that the present invention is applicable to other types of absorbent garments and absorbent articles. Thus, the description of unique features of the invention in conjunction with a feminine hygiene article is equally applicable to the use of the invention with other types of absorbent garments and articles, such as baby diapers and adult incontinence garments. A depiction of an embodiment of the present invention in the form of a diaper is provided in FIG. 7.

FIG. 1 is a planar view of an embodiment of the present invention depicted in the fully-flattened position, with the topsheet partially cut-away to show the interior of the garment. The article 10 is comprised of an absorbent core 12 encased between a topsheet 14 and a backsheet 16.

The topsheet 14 and backsheet 16 may be constructed from a wide variety of materials known in the art. The invention is not intended to be limited to any specific materials or constructions of these components. In a preferred embodiment, the , topsheet 14 and backsheet 16 form the outer layers of the present invention, and are shaped and sized according to the requirements of each of the various types of absorbent garment.

The backsheet 16 can be made of any suitable pliable liquid impervious material known in the art or later discovered. Typical backsheet materials include films of polyethylene, polypropylene, polyester, nylon, and polyvinyl chloride and blends of these materials. For example, the backsheet 16 can be comprised of a pigmented polyethylene film having a thickness in the range of 0.02-0.04 mm. The backsheet 16 may also be comprised of several layers of material that are partly or wholly overlaid on one another to form a laminate structure.

In addition, the backsheet 16 may be covered with a fibrous, nonwoven fabric such as is disclosed, for example, in U.S. Pat. 4,646,362 issued to Heran et al., the disclosure of which is hereby incorporated by reference in its entirety, and in a manner consistent with the present application and invention. Materials for such a fibrous nonwoven fabric outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene, polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the backsheet 16 may comprise three panels wherein a central backsheet 16 panel is positioned adjacent the storage layer while outboard non-woven breathable side backsheet 16 panels are attached to the side edges of the central backsheet 16 panel. The backsheet 16 may be formed from materials that provide added breathability, such as a microporous coverstock and the like.

The moisture-pervious topsheet 14 can be made of any suitable relatively liquid-pervious material currently known in the art or later discovered that permits passage of a liquid therethrough. Non-woven materials are exemplary because such materials readily allow the passage of liquids to the underlying absorbent core 12. Examples of suitable topsheet 14 materials include non-woven spun-bonded or carded webs of polypropylene, polyethylene, nylon, polyester and blends of these materials.

The topsheet 14 may also be made of single-ply nonwoven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a topsheet material for the given application.

The topsheet 14 may be comprised of several regions having different properties.

In one embodiment of the present invention, the laterally distal portions of the topsheet 14 are preferably substantially liquid-impervious and hydrophobic. Different properties, such as liquid perviousness and hydrophilicity, may be imparted upon the topsheet 14 by treating the topsheet 14 with adhesives, surfactants, or other chemicals, or by other means. The topsheet 14 may also be comprised of several layers of material that are partly or wholly overlaid on one another to form a laminate structure.

The backsheet 16 and the topsheet 14 are “associated” with one another. The term “associated” encompasses configurations whereby the topsheet 14 is directly joined to the backsheet 16 by affixing the topsheet 14 directly to the backsheet 16, and ; t configurations whereby the topsheet 14 is indirectly joined to the backsheet 16 by affixing the topsheet 14 through intermediate members, which in turn are affixed to the backsheet 16. While the backsheet 16 and topsheet 14 in the preferred embodiment have substantially the same dimensions, they may also have different dimensions.

In a preferred embodiment, the topsheet 14 and backsheet 16 are substantially the same shape and size. Also in a preferred embodiment, the topsheet 14 and backsheet 16 are roughly rectangular in shape, with the long dimension of the rectangle being in the longitudinal direction 102 and the short dimension of the rectangle in the lateral direction 100. The topsheet 14 and backsheet 16 are preferably continuously joined to each other around their respective peripheral edges using hot-melt adhesives or ultrasonic bonding to form a liquid impervious seam 18 around the edge of the article 10. However, the seam 18 may also be intermittent, or otherwise may not provide a liquid impermeable seal. In another preferred embodiment, the topsheet 14 and backsheet 16 are joined to one another tightly around the absorbent core 12 to retain the absorbent core 12 in place within the article 10, eliminating the need to join the absorbent core 12 to the topsheet 14 or the backsheet 16. Those skilled in the art are capable of joining topsheet 14 to backsheet 16 using any desirable configuration, and any desirable mechanism of joining the respective sheets.

Also in a preferred embodiment, the topsheet 14 and backsheet 16 further comprise a wing 20 located on each lateral edge of the article 10. These wings may be wrapped around the garment of the user to secure the article 10 to the user's garment and to prevent soiling of the body and clothing by body fluids. The article 10 may also comprise one or more adhesive strips (not shown) to attach the article 10 to the user's garment. Such wings 20 and adhesive strips are disclosed in U.S. Pat. No. 4,687,478 issued to Van Tillburg, the disclosure of which is incorporated by reference herein in its entirety in a manner consistent with the present invention.

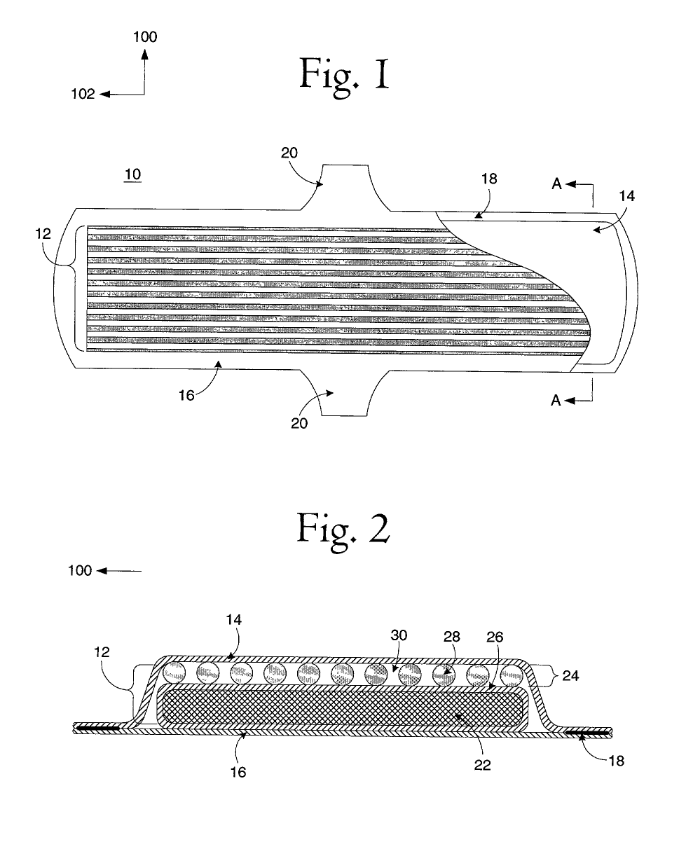

Referring now to FIG. 2, which is a cross-sectional view of the article 10 of FIG. 1, as viewed across reference line A-A, the absorbent core 12 is comprised of a storage layer 22 and an acquisition layer 24. The acquisition layer 24 preferably is located between the topsheet 14 and the storage layer 22.

Generally, the storage layer 22 serves to contain the fluids that enter the article 10. Ideally, the storage layer 22 will not release absorbed fluids during normal use, so that the user remains dry and comfortable. The storage layer 22 may have a variety of shapes and sizes, depending on the shape, size, and absorbency requirements of the particular type of absorbent garment into which the storage layer 22 is incorporated. Generally, however, the storage layer 22 is roughly rectangular with the long dimension of the storage layer 22 being roughly parallel with the longitudinal axis 102 of the article 10. The longitudinal dimension 102 of the storage layer 22 is typically less than that of the topsheet 14 and the backsheet 16. The storage layer 22 may be attached to the backsheet 16 by means of ultrasonic bonding, hot melt adhesives, or other means known to the art. Alternatively, the storage layer 22 may be placed in the article 10 without attaching it to the backsheet 16.

In a preferred embodiment of the present invention the storage layer 22 uses a super-absorbent polymer to contain fluids. Super-absorbent polymers are well-known in the art, and generally include materials that are able to absorb many times their own weight in liquid. Super-absorbent polymers may be used in a variety of forms, including, but not limited to: particulate, flaked, granular, or powdered forms. Super-absorbent polymer materials generally fall into three classes: starch graft copolymers, cross-linked carboxymethylcellulose derivatives, and modified hydrophilic polyacrylates. Exemplary SAP materials include, but are not limited to: carboxylated cellulose, hydrolyzed acrylonitrile-grafted starch, acrylic acid derivative polymers, polyacrylonitrile derivatives, polyacrylamide-type compounds, and saponified vinyl acetate/methyl acrylate copolymers. The manufacture of hydrogel-forming polymer materials is disclosed in U.S. Reissue Pat. No. 032,649, issued to Brandt et al., the disclosure of which is herein incorporated by reference in its entirety in a manner consistent with the present invention.

The super absorbent material can be mixed with other materials known in the art, preferably fibrous materials, to form a liquid-absorbent matrix. These additional materials include, but are not limited to, hydrophilic fibrous materials such as cellulose fibers, modified cellulose fibers (e.g., comminuted wood pulp or internally or externally cross-linked cellulose fibers), rayon, polyester fibers such as polyethylene terephthalate (DACRON™), hydrophilic nylon (HYDROFIL™), and hydrophilized hydrophobic fibers (e.g., surfactant- or silica-treated thermoplastic fibers derived, for example, from polyolefins).

In one preferred embodiment, the storage layer 22 is formed from a very thin, high density, folded absorbent laminate that contains SAP and stabilization additives. The storage layer 22 of this embodiment comprises from about 50 to about 95 percent by weight, preferably from about 60 to about 95 percent by weight, and more preferably from about 70 percent to about 90 percent by weight SAP. The storage layer 22 also comprises an effective amount of at least one fibrous or particulate additive for maintaining a high SAP efficiency of the storage layer 22 of at least about 70 percent, preferably at least about 80 percent, and more preferably at least about 85 percent.

The folded structure of the storage layer 22 may be formed by folding at least once the marginal sides of the storage layer 22 to form a central channel extending longitudinally along the storage layer 22. The central channel may increase the free volume that is available for temporarily containing large doses of liquid such as urine and body wastes, until they can be absorbed by the SAP in the laminate, thereby improving the surge handling ability of the article 10. The folded structure of the storage layer 22 also increases the effective surface area of laminate that is available for liquid absorption. Thin, high density, folded laminate storage layers 22 are more fully described U.S. Pat. No. 6,068,620, issued to Chmielewski, the disclosure of which is herein incorporated by reference in its entirety for all purposes and in a manner that is consistent with this application and invention.

In another preferred embodiment, the storage layer 22 is conventional composite of SAP and fluff of cellulosic fibers, such as wood pulp fibers, cotton linters, and the like. The storage layer 22 may be formed in a known manner, as by a pocket-forming process from cellulosic fibers, and from distributed particles of a super-absorbent polymer, which is preferably a surface cross-linked super absorbent polyacrylate. The storage layer 22 may further comprise pockets or storage cells of SAP, or a multi-layered laminate of SAP-bearing layers and conventional fluff pulp layers. Such constructions are known in the art, and disclosed in U.S. Pat. No. 5,863,288, issued to Baker, the disclosure of which is herein incorporated by reference in its entirety in a manner consistent with the present invention. The storage layer 22 may also have differing properties in the central and periphery regions of the storage layer 22, as disclosed in U.S. Pat. No. 5,330,456 issued to Robinson, the disclosure of which is incorporated herein by reference in its entirety in a manner consistent with the present invention.

Still referring to FIG. 2, the storage layer 22 is preferably encased in a tissue wrap 26, which may fully or partly envelop the storage layer 22. The tissue wrap 26 serves to stabilize the storage layer 22 by helping the storage layer maintain its shape and retaining any loose particles of SAP, particulate additives, fibers, or any other material comprising the storage layer 22. Tissue wrap 26 materials are well-known in the art, and are often made from synthetic fiber webs or airlaid fluff pulp. The tissue wrap 26 is typically the same length (i.e., in the longitudinal dimension 102) as the storage layer 22, although it may be slightly longer or shorter.

In the present invention, the upper and lower portions (i.e. those nearest the topsheet 14 and backsheet 16 respectively) of the tissue wrap 26 may be made from separate pieces of different material, or from one or more pieces of the same material. Preferably, the upper portion of the tissue wrap 26 has an effective porosity for allowing the easy passage of liquid and for preventing the migration of SAP into the acquisition layer 24 and topsheet 14 area of the article 10. The lower portion of the tissue wrap 26 is preferably substantially liquid impervious.

The lower portion of tissue wrap 26 may be made less liquid pervious by, for example, using a denser tissue material. The lower portion of the tissue wrap 26 may also be rendered liquid impervious by using a liquid impervious material, by applying adhesive to a liquid pervious material, or by other chemical or physical treatment of a liquid-pervious material, as is known in the art. In one embodiment of the present invention a single piece of generally liquid pervious material is rendered liquid impervious on one side of one half by treating that part of the material with preferably about 6 g/m², of a hydrophilic adhesive at high coverage from, for example, a CONTROL COAT™ or DYNAFIBER™ UFD-17 spray nozzle. The treated half is adhered to the bottom of the storage layer 22 to form the bottom portion of the tissue wrap 26, and the untreated half is wrapped around the storage layer 22 to form the upper portion of the tissue wrap 26. Those skilled in the art will appreciate that there are numerous methods of modifying the physical properties of the tissue wrap 26 to obtain various benefits.

The tissue wrap 26 may be attached to the backsheet 16 by ultrasonic bonding, hot melt adhesive, or any other suitable means. Alternatively, the tissue wrap 26 may be placed in the article 10 without attaching it to the backsheet 16.

Still referring to FIG. 2, the acquisition layer 24 comprises a plurality of yarns 28 having channels 30 between them. In a preferred embodiment, the yarns 28 and channels 30 extend for substantially the entire length of the storage layer 22, and are distributed across the entire width of the storage layer 22. Together, the yarns 28 and channels 30 create a bimodal acquisition layer 24. One mode of fluid acquisition is provided by the yarns 28, and the other mode is provided by the acquisition channels 30. The yarns 28 also provide a physical separation between the topsheet 14 and the storage layer 22, and they transmit fluid from the topsheet 14 to the storage layer 22 to prevent rewet. The channels 30 are generally empty, and provide a conduit in which fluids may freely flow to the longitudinal ends of the article 10. Fluids in the channels 30 are absorbed directly into the storage layer 22.

The yarns 28 may be comprised of fibers that are woven into yarns. These woven yarns 28 typically will not unravel or become separated during manufacturing or use, so there is no need for additional tissue or other wrapping to be placed around the yarns 28 to prevent them from separating into individual fibers and becoming less effective. This simplified construction allows the yarns 28 to be integrated with existing storage layers with little or no modification to the design of the storage layer 22 or the topsheet 14. Furthermore, the yarns 28 generally extend in the machine direction of the article 10, which simplifies and minimizes the number of additional operations that are necessary to make the article 10.

A further cost benefit is obtained in transportation costs. Traditional acquisition layers use high-volume, low basis weight material, which is relatively expensive to transport because it occupies a large volume of space. Yarns 28, on the other hand, may be transported to the article manufacturer packed tightly together in spools or otherwise. The yarns 28 used to make an acquisition layer 24 may occupy less than half the volume required by a traditional acquisition layer 24 material. Using yarns 28 to form the acquisition layer 24 provides a construction cost benefit because the relatively low volume yarns 28 cost less to transport, per unit of production, than traditional high-volume, low basis weight material.

It is desirable for the acquisition layer 24 to provide the article 10 with a high surge handling capacity. A high surge handling capacity reduces the likelihood that the article 10 will be saturated and begin leaking. Furthermore, by redistributing the fluids to the longitudinal ends of the article 10 the SAP efficiency improves because portions of the garment that would not normally be wetted by the fluid will now be exposed to fluid. Because the article 10 is typically much longer than it is wide, the channels 30 and yarns 28 must accelerate the fluid flow in the longitudinal direction, and retard the flow in the lateral direction. The channels 30 and yarns 28 work together to provide a high surge handling capacity, redistribute fluids rapidly in the longitudinal direction and slow the natural fluid flow in the lateral direction.

The surge handling capacity of the article 10 is partly a function of the acquisition rate of the yarns 28, and partly a function of the volume of free space in the acquisition channels 30. The acquisition rate of the yarns 28 is the rate at which the yarns 28 absorb fluids, which is a function of the material properties of the yarns 28. The acquisition rate of the channels 30 is simply the rate at which fluids fill the empty space of the channels 30. Generally, the acquisition rate of the channels 30 will be greater than that of the yarns 28. In addition, the surge handling capacity of acquisition layer 24 is often correlated to the bulk of the layer. The yarn-and-channel construction of the present invention provides a higher equivalent bulk than traditional acquisition layers, thereby providing improved surge handling capacity.

As the total volume of the acquisition channels 30 increases, that is, as the free space between the yarns 28 increases, the surge capacity increases. However, if the channels 30 become too wide, then the channels 30 may deform or collapse, which may reduce their volume and thereby impair their effectiveness. The volume of the acquisition channels 30 may be increased by using relatively few yarns 28, each having a relatively large diameter, while simultaneously increasing the channel width, thereby creating taller channels 30 that have greater volume and are less likely to collapse, however, this leads to a thicker absorbent core 12, which may be less comfortable. To overcome this problem, several different size yarns 28 and channels 30 widths may be used. In one embodiment, the yarns 28 in the center region are thicker than those along the sides of the article 10, and the channels 30 in the center region are wider than those along the edges.

The rewet of the garment may be modified by changing the diameters of the yarns 28 and the widths of the acquisition channels 30. The rewet resistance of the garment may be improved by increasing the ability of the yarns 28 and channels 30 to drain the topsheet, and the ability of the storage layer 22 to drain the yarns 28 and channels 30. The draining ability of a substance is generally inversely proportional to the substance's pore size, with materials having a smaller pore size being better suited to drain fluids from those having a larger pore size. The pore size of the yarns 28 is generally a function of how closely the fibers of the yarns 28 are packed together, and the pore size of the channels 30 is generally equal to their width.

The pore sizes of the yarns 28 and channels 30 are preferably chosen to effectively drain fluid from the topsheet 14 to the storage layer 22. The operation of the invention and the relationship between the various parts and their respective pore sizes may be demonstrated by the following hypothetical description of the operation of an embodiment of the present invention.

In a hypothetical model of operation, a portion of the fluid entering the garment 10 is distributed into the topsheet 14, and a portion of the fluid is forced, by hydraulic or mechanical pressure, through the topsheet 14 and into the channels 30. The fluid in the channels 30 may be distributed along the length of the channels 30, and is drained into the storage layer 22, which is selected to have a smaller pore size than the channels 30. A portion of the fluid in the channels 30 may also be drained into the yarns 28, which are also selected to have a smaller pore size than the channels 30. The fluid that is initially distributed into the topsheet 14 is also drained into the yarns 28, which are selected to have a smaller pore size than the topsheet 14. The storage layer 22 is selected to have a smaller pore size than the yarns 28, and so the fluid entering the yarns 28 from the topsheet 14 and channels 30 is also drained into the storage layer 22. Rewet is generally prevented because the yams 28 and channels 30 have a greater pore size than the storage layer 22, thereby preventing drainage of fluid away from the core. This hypothetical model of the operation of the present invention is for illustrative purposes only, and is not intended to limit the scope of the present invention in any way. Other models may also be used to describe the operation of the invention.

The channels 30 may also be designed to partly accommodate swollen SAP, which may press upwards into the channels 30 after absorbing fluids and remain in the channels 30 until the fluids are distributed to lower portions of the storage layer 22. Ideally, the channels 30 are large enough to accommodate some swelling, but the swollen SAP will not interfere with the ability of the channels 30 to transfer fluid along the length of the article 10.

It is therefore important to design the yarns 28 and acquisition channels 30 such that the article 10 is comfortable and provide improved rewet resistance, but that the channels 30 do not collapse in use. In a preferred embodiment, the yarns 28 have a diameter of about 1 millimeter to about 6 millimeters. In a more preferred embodiment, the yarns 28 may have a diameter of about 2 millimeters to about 5 millimeters. In still another embodiment, the yarns 28 may have a diameter of about 3 millimeters to about 4 millimeters. In various embodiments, the channels 30 have a width of about 1 millimeter to about 6 millimeters, or more preferably a width of about 2 millimeters to about 5 millimeters. The channels 30 may also have a width of about 3 millimeters to about 4 millimeters

Referring back to FIG. 1, in a preferred embodiment, channels 30 and yarns 28 are oriented roughly parallel to the longitudinal axis 102 of the article 10 to promote flow in the longitudinal direction. Such a construction also retards fluid flow in the lateral direction because the yarns 28 act as a physical barrier to lateral flow.

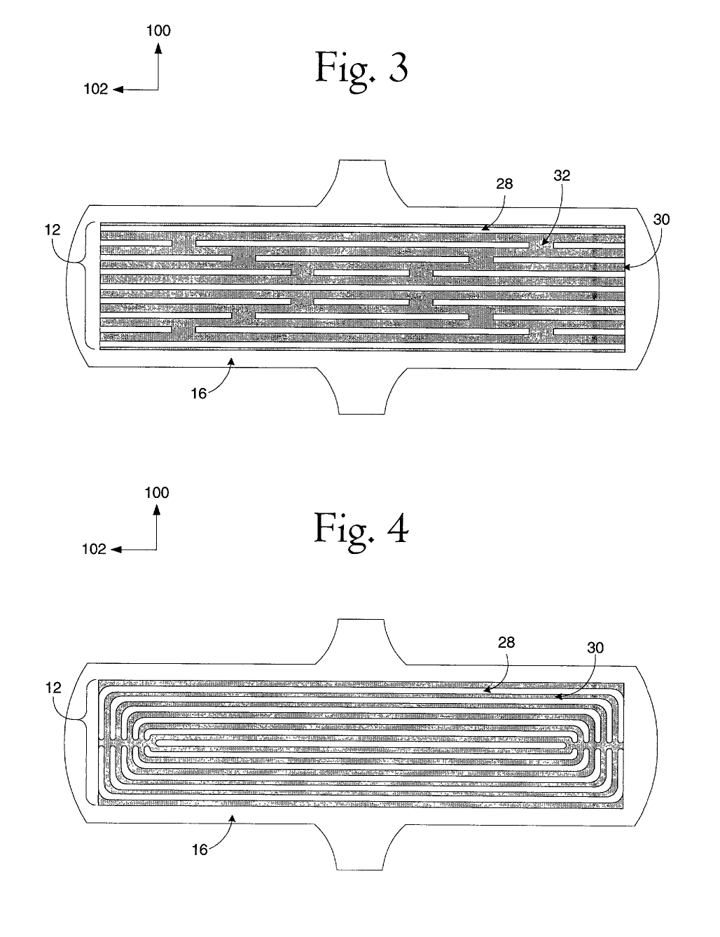

Other configurations may also be employed to promote fluid flow to all parts of the article 10. FIGS. 3 and 4 are views of two embodiments of the present invention with the topsheet removed. In the embodiment of FIG. 3, the yarns 28 are oriented roughly parallel to the longitudinal axis 102 of the article 10, to promote longitudinal fluid flow and retard lateral flow, and there are openings 32 in the yarns 28 that allow fluid to flow from one channel 30 to the next, and towards the comers of the storage layer 22. With such a configuration, fluids may be distributed in a substantially rectangular pattern to make full use of the absorbent article 10. Other configurations of openings 32 may also be employed to allow fluid to pass from one channel 30 to the next and to promote controlled lateral flow of fluid.

FIG. 4 depicts an embodiment of the present invention in which the ends of the yarns 28 are curved inward at the longitudinal ends of the article 10, creating a series of concentric ring-shaped structures, to prevent leakage out of the front and back of the article 10 during use.

The yarns 28 may be made from any number of natural or synthetic materials. For example, the yarns 28 may be made from various types of polyethylene, polyester, polyurethane or polyacrylic synthetics. The yarns 28 may also be made from capillary channel fibers, which are synthetic fibers that are engineered to have channels 30 that promote capillary flow of fluids along their length. Such fibers are disclosed in U.S. Statutory Invention Registration No. H1511, which was previously incorporated herein by reference. In such an embodiment, the capillary channel fibers assist the acquisition channels 30 in redistributing material from the insult point to the longitudinal ends of the article 10 by means of highly promoted capillary action.

Each individual yarn may have properties that are individually tailored, based on each yarn's location in the article 10, to provide the greatest overall benefit to the article 10. The properties of each of the yarns 28 may be manipulated by several means, including, but not limited to: altering the material composition, adjusting the weave density, or chemical treatment. These and other means may be used singly and in combination with one another. Those skilled in the art are capable of individually tailoring each yarn 28 to provide the greatest overall benefit, using the guidelines provided herein.

In a preferred embodiment, the yarns 28 in the center region have a high acquisition rate to assist in surge handling, and the yarns 28 in the edge regions have high wicking properties to draw fluids away from the center region and transmit the fluids along the length of the garment. These properties may be obtained by at least two separate means, or by a combination of means, which are described as follows.

A first means of obtaining this preferred embodiment is to provide yarns 28 in the center region of the acquisition layer 24 having a relatively high fluid acquisition rate, and to provide the yarns 28 in the edge regions having relatively greater wicking properties.

Alternatively, or in conjunction with the first means, a second means of obtaining this preferred embodiment is to provide yarns 28 in the center region that are comprised of a relatively loose weave of fibers, and to provide the yarns 28 in the edge regions that are comprised of a relatively dense weave of fibers. If the yarns are hydrophilic, then the loosely woven yarns 28 in the center region, may provide a high acquisition rate, and the densely woven fibers in the edge yarns 28 provide increased capillary action within the structure of the weave, leading to better wicking properties.

The denier of the yarns may be selected to provide various benefits, as described herein, and as will be appreciated by those skilled in the art. In one embodiment, the yarn denier may be about 1000 to about 6000. In another embodiment, the yarn denier may be about 2000 to about 5000. In still another embodiment, the yarn denier may be about 4100 to about 4150. The fibers comprising the yarns may also be selected to have various deniers to provide various benefits, as understood by those skilled in the art. In one embodiment, the fiber denier may be about 1.00 to about 14.00. In another embodiment, the fiber denier may be about 5.00 to about 10.00. In still another embodiment, the fiber denier may be about 7.00 to about 7.25.

It is important that article 10 be comfortable to the user after it has absorbed fluids. To this end, the yarns 28 may be selected to provide good transport properties to move fluid from the topsheet 14 to the storage layer 22.

Furthermore, In another preferred embodiment, the yarns 28 in the side regions may be selected to provide additional wet strength to the edge regions of the absorbent core 12, thereby helping to stabilize the storage layer 22 in use. The yarns 28 in the side regions may also be infused with a skin care product to provide additional comfort to the user in the edge portions of the article 10, which may be pressed tightly against the user's skin.

Referring back to FIG. 2, the yarns 28 may be attached to the tissue wrap 26, the topsheet 14, or both, to hold the yarns 28 in their proper positions and to ensure that the yarns 28 remain in physical contact with the tissue wrap 26. If no tissue wrap 26 is used, then the yarns 28 may be attached directly to the storage layer 22. The attachments may be made of adhesives, stitching, ultrasonic bonding, or any other means known in the art or later developed. It is important for fluids captured in the yarns 28 to be transmitted to the storage layer 22. The connection means for attaching yarn 28 to tissue wrap 26 or storage layer 22 should not create a liquid impervious layer between the yarns 28 and the storage layer 22.

In a preferred embodiment, the yarns 28 are attached to the tissue wrap 26 or storage layer 22 using lines of hydrophilic adhesive (not shown), in an amount of about 2.0 g/m² or less, that extend along the entire length of each yarn. This is a generally low coverage adhesive application that maintains the liquid permeability of the yarns 28 and the surface to which they are attached. The adhesive preferably is applied using a CONTROLLED FIBERIZATION™ nozzle or DYNAFIBER™ UFD-5. Of course it should be understood that the yarns 28 may be attached to the topsheet 14, tissue wrap 26, or storage layer 22 directly or indirectly through intermediate optional laminates or layers. In another embodiment of the present invention, the yarns 28 are attached to the tissue wrap 26 by periodic spots of adhesive, rather than continuous lines of adhesive as described above.

FIG. 5 depicts a cross-section of another embodiment of the present invention in which the tissue wrap 26 envelops both the storage layer 22 and the acquisition layer 24. Such an embodiment may provide an economic benefit to the article manufacturer. In this embodiment, adhesive is placed on the interior surface of the tissue wrap 26 prior to it being wrapped around the storage layer 22 and acquisition layer 24, much like it may be applied if the tissue wrap 26 enveloped only the storage layer 22. The tissue wrap 26 then is folded over the storage layer 22 and acquisition layer 24, binding them in place. This construction may eliminate the need for separate lines of adhesive for holding the yarns 28 in place.

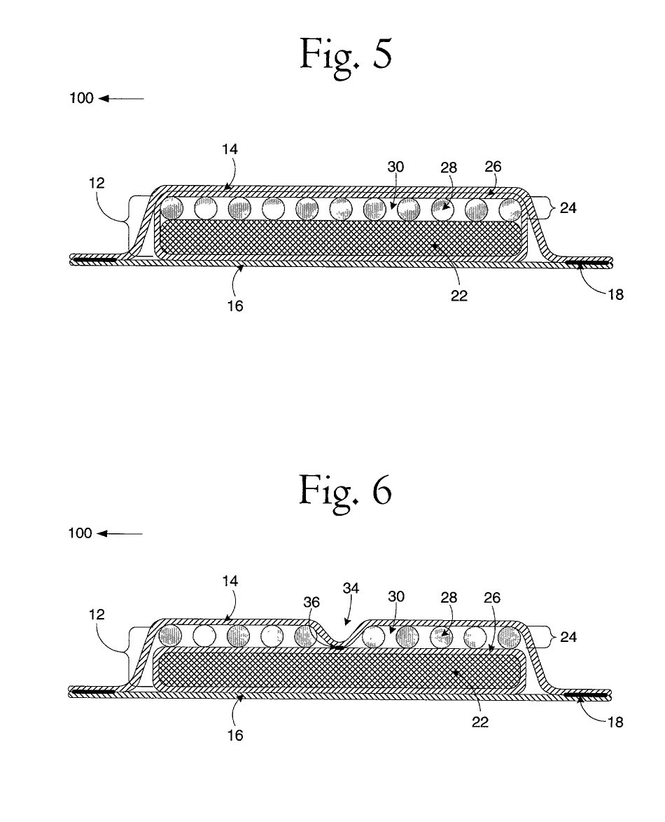

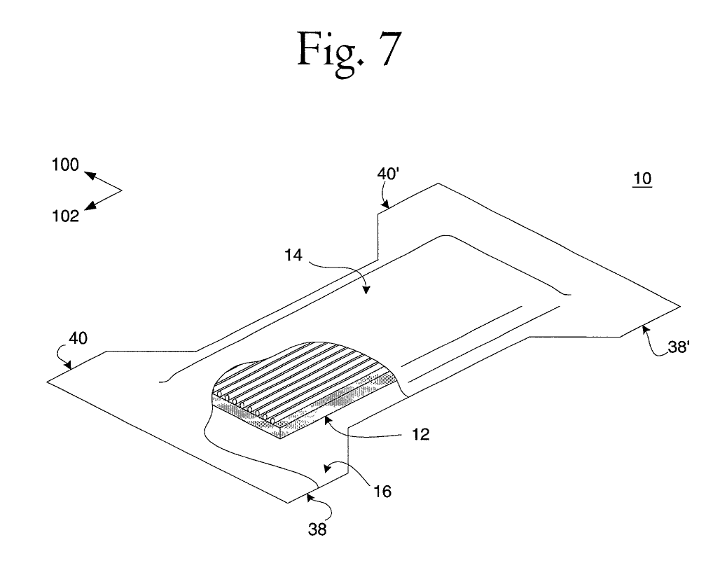

Referring now to FIG. 6, which is a depiction of a cross-section of another embodiment of the present invention, the topsheet 14 may also be attached to the tissue wrap 26 or the storage layer 22 in all or part of the length of one or more of the acquisition channels 30. In such an embodiment, the external channel 34 acts to distribute fluids along the length of the article 10, much like the acquisition channels 30 do. The fluid need not penetrate the topsheet 14 before proceeding along the external channel 34, which allows faster longitudinal redistribution. However, the external channel 34 may not capture the fluid within the garment and isolate the fluid from the user as well as the acquisition channels 30. Also, because the topsheet 14 is closer to the storage layer 22 in this embodiment, rewet may be more likely in the regions surrounding the attachment 36 of the topsheet 14 to the tissue layer 26 or storage layer 22. In a preferred embodiment, one or more external channels 34 exist only in the central region of the article 10 to supplement the acquisition channels 30, and do not extend for the full length of the article 10 along its longitudinal dimension.

As noted elsewhere herein, the present invention may be used with any type of absorbent article, and is not intended to be limited to the uses depicted in the discussed embodiments. FIG. 7 depicts an embodiment of the present invention in which the garment 10 is formed in the shape of a diaper. In FIG. 7 the absorbent core 12 is disposed between a topsheet 14 and a backsheet 16 of a diaper. The garment 10 is depicted in the fully flattened position with the side seams 38, 38′, 40, 40′ separated. For use, the garment is manipulated to form a garment having two leg holes and a waist hole by joining seam 38 to seam 38′, and joining seam 40 to seam 40′. The details of such constructions are known in the art.

Other embodiments, uses, and advantages of the invention will be apparent to those skilled in the art from consideration of the specification and practice of the invention disclosed herein. The specification should be considered exemplary only, and the scope of the invention is accordingly intended to be limited to the following claims.

We claim:

1. An absorbent article having a longitudinal dimension corresponding to the fore-aft direction of a wearer's body and a lateral dimension corresponding to the side-to-side direction of a wearer's body, comprising:
a liquid permeable topsheet;
a liquid impermeable backsheet; and
an absorbent core positioned between the topsheet and the backsheet, the absorbent core comprising:
an acquisition layer comprised of at least two yarns having an acquisition channel between each adjacent pair of yarns; and
a storage layer;
wherein the storage layer is positioned between the acquisition layer and the backsheet.

2. The absorbent article of claim 1 wherein the at least two yarns are oriented substantially parallel with the longitudinal dimension of the article.

3. The absorbent article of claim 2 wherein at least one of the yarns contains at least one opening to permit fluid to flow there through.

4. The absorbent article of claim 3, wherein the opening is placed substantially along a diagonal axis of the acquisition layer.

5. The absorbent article of claim 1 wherein the at least two yarns are oriented in a series of concentric rings, each of the rings having a long axis and a short axis, the long axis being oriented substantially parallel with the longitudinal dimension of the article.

6. The absorbent article of claim 5, further comprising one or more passages in the perimeter of each ring shape to allow the passage of fluid there through.

7. The absorbent article of claim 1 wherein the acquisition channels provide free volumes for accommodating surges of fluid and transferring the fluids substantially along the longitudinal dimension of the article.

8. The absorbent article of claim 1 wherein the yarns retard the lateral flow of fluid.

9. The absorbent article of claim 1 wherein the topsheet, plurality of yarns, acquisition channels, and storage layer are selected to transfer fluid from the topsheet to the storage layer, while retarding the opposite flow of fluid.

10. The absorbent article of claim 1 wherein the at least two yarns comprise:
at least one edge yarn located near side edges of the absorbent core; and
at least one center yarn located near a center region of the absorbent core and having different physical properties than the at least one edge yarn.

11. The absorbent article of claim 10 wherein the at least one edge yarn provides additional wet strength to lateral edges of the absorbent core.

12. The absorbent article of claim 10 wherein the at least one edge yarn contains skin care products.

13. The absorbent article of claim 10 wherein the at least one edge yarn has greater wicking properties than the at least one center yarn.

14. The absorbent article of claim 10 wherein the at least one center yarn has greater acquisition properties than the at least one edge yarn.

15. The absorbent article of claim 1 wherein each of the yarns has a diameter of about 1 millimeters to about 6 millimeters.

16. The absorbent article of claim 1 wherein each of the yarns has a diameter of about 2 millimeters to about 5 millimeters.

17. The absorbent article of claim 1 wherein each of the yarns has a diameter of about 3 millimeters to about 4 millimeters.

18. The absorbent article of claim 1 wherein each of the acquisition channels has an average width of about 1 millimeter to about 6 millimeters.

19. The absorbent article of claim 1 wherein each of the acquisition channels has an average width of about 2 millimeters to about 5 millimeters.

20. The absorbent article of claim 1 wherein each of the acquisition channels has an average width of about 3 millimeters to about 4 millimeters.

21. The absorbent article of claim 1 wherein one or more yarns has a diameter different from the diameter of one or more other yarns.

22. The absorbent article of claim 21 wherein the yarns in the center region have a greater diameter than the yarns in the side regions.

23. The absorbent article of claim 1 wherein one or more of the acquisition channels has a width different from one or more of the other acquisition channels.

24. The absorbent article of claim 23 wherein the acquisition channels in the center region are wider than the acquisition channels in the side regions.

25. The absorbent article of claim 1 wherein the yarns are attached to the storage layer by hot melt adhesive glue along at least a portion of each yarn's length.

26. The absorbent article of claim 1 wherein one or more of the yarns is made from a material selected from the group consisting of polyethylene, polyester, polyurethane and polyacrylic synthetics.

27. The absorbent article of claim 1 wherein each of the fibers comprising each yarn has a fiber denier of about 1.00 to about 14.00.

28. The absorbent article of claim 1 wherein each of the fibers comprising each yarn has a fiber denier of about 5.00 to about 10.00.

29. The absorbent article of claim 1 wherein each of the fibers comprising each yarn has a fiber denier of about 7.00 to about 7.25.

30. The absorbent article of claim 1 wherein each of the yarns has a yarn denier of about 1000 to about 6000.

31. The absorbent article of claim 1 wherein each of the yarns has a yarn denier of about 2000 to about 5000.

32. The absorbent article of claim 1 wherein each of the yarns has a yarn denier of about 4100 to about 4150.

33. The absorbent article of claim 1, wherein the storage layer comprises a composite of fibers and super-absorbent polymer.

34. The absorbent article of claim 1, further comprising an at least partly liquid permeable tissue wrap surrounding the storage layer.

35. The absorbent article of claim 34 wherein the topsheet is operably attached to the at least partly liquid permeable tissue wrap in one or more of the acquisition channels, creating one or more topsheet channels.

36. The absorbent article of claim 1, further comprising an at least partly liquid permeable tissue wrap surrounding the absorbent core.

37. The absorbent article of claim 1, wherein the topsheet is operably attached to the backsheet around the perimeter of the article and the absorbent core is held in place without being bonded to either of the topsheet or the backsheet.

38. The absorbent article of claim 1 wherein the absorbent article is a feminine care product.

39. The absorbent article of claim 1 wherein the absorbent article is a diaper.

40. The absorbent article of claim 1 wherein the absorbent article is an adult incontinence product.
